SEQUENCE LISTING (6 sequences)

SEQ ID NO: 1 - DNA, 29 bp, Artificial Sequence
  VLP recognition element
gggaatcacatcagtaactgagtgacttc

SEQ ID NO: 2 - DNA, 2862 bp, Artificial Sequence
  Cas13d
atgggcaaaaaaatccatgcacgtgatctgcgtgaacagcgtaaaaccgatcgtaccgaaaaatttgcagaccagaacaaaaaacgtgaagcagaacgtgcagttccgaaaaaagatgcagcagttagcgttaaaagcgttagcagcgtgagcagcaaaaaagataatgttaccaaaagcatggcaaaagcagccggtgtgaaaagcgtttttgcagttggtaataccgtttacatgaccagctttggtcgtggtaatgatgcagttctggaacagaaaattgttgataccagccatgaaccgctgaatattgatgatccggcatatcagctgaatgtggttaccatgaatggttatagcgttaccggtcatcgtggtgaaaccgttagcgcagttaccgataatccgctgcgtcgttttaatggtcgtaaaaaagacgaaccggaacagagcgttccgaccgatatgctgtgtctgaaaccgacactggaaaaaaagttttttggcaaagagttcgacgacaacattcatatccagctgatttataacatcctggacatcgaaaaaatcctggcagtttatagcaccaatgcaatttatgccctgaataacatgagcgcagatgaaaacattgagaacagcgacttttttatgaaacgcaccaccgatgaaaccttcgatgatttcgaaaaaaagaaagaaagcaccaacagccgtgaaaaagcagattttgatgccttcgaaaaattcatcggtaattatcgcctggcctattttgcagatgccttttatgtcaacaaaaagaacccgaaaggcaaagccaaaaatgtgctgcgtgaagataaagaactgtatagcgttctgaccctgattggtaaactgcgtcattggtgtgttcatagcgaagaaggtcgcgcagaattttggctgtataaactggatgaactgaaggacgatttcaaaaacgttctggatgtggtttataatcgtccggtggaagaaattaacaaccgctttatcgaaaacaacaaagtgaacattcagattctgggcagcgtgtataaaaacaccgatattgcagaactggtgcgcagctattatgaatttctgatcaccaaaaaatacaaaaacatgggctttagcatcaaaaaactgcgtgaaagcatgctggaaggtaaaggttatgccgataaagaatatgatagcgtgcgcaacaaactgtatcagatgaccgattttattctgtacaccggctatatcaacgaagatagcgatcgtgcagatgatctggttaataccctgcgtagcagcctgaaagaagatgataaaaccaccgtgtattgcaaagaggcagattacctttggaaaaagtatcgcgaaagcattcgtgaagttgccgatgcactggatggtgacaatattaagaaactgagcaaaagcaacatcgagatccaagaggataaactgcgcaaatgctttattagctatgcagatagcgttagcgaatttaccaaactgatttatctgctgacccgttttctgagcggtaaagaaatcaacgatctggtgaccacactgatcaacaaatttgataacattcgcagctttctggaaatcatggatgaattaggtctggatcgtacctttaccgcagaatatagcttttttgaaggcagcaccaaatatctggccgaactggtggaactgaatagctttgttaaatcctgcagcttcgatattaatgccaaacgtaccatgtatcgtgatgccctggatattctgggtattgaatcagataaaaccgaagaggatatcgagaaaatgatcgacaatatcctgcagattgatgccaacggtgataaaaagctgaaaaaaaacaatggcctgcgcaactttattgccagcaatgttattgatagcaaccgcttcaaatacctggtgcgttatggtaacccgaagaaaattcgtgaaaccgcaaaatgtaaaccggcagttcgttttgtgctgaatgaaattccggatgcacagatcgaacgttattatgaagcatgttgtccgaaaaataccgcactgtgtagcgcaaataaacgtcgtgaaaaactggcagatatgatcgccgaaatcaaattcgaaaacttttccgatgcaggcaactatcagaaagcaaatgtgaccagccgtaccagcgaagcagaaattaaacgtaaaaaccaggccattattcgcctgtatctgaccgttatgtatatcatgctgaaaaacctggtgaatgtgaatgcccgttatgtgattgcatttcattgtgttgaacgcgacacaaaactgtatgccgaaagcggtctggaagtgggtaatattgaaaagaataaaaccaacctgaccatggccgttatgggtgttaaactggaaaatggcattatcaaaaccgagttcgataagagctttgcagaaaatgcagcaaatcgttatctgcgtaatgcgcgttggtataagctgattctggataatctgaagaaaagcgaacgtgccgttgtgaatgaatttcgcaataccgtgtgtcatctgaatgccattcgcaacattaacatcaacattaaagagatcaaagaggtcgagaactatttcgccctgtatcattatctgatccagaaacatctggaaaaccgcttcgcagacaaaaaagttgaacgtgataccggtgatttcatcagcaaactggaagaacataaaacctactgcaaagacttcgtgaaagcctattgtaccccgtttggttataatctggttcgctataaaaacctgacgatcgatggtctgttcgacaaaaactatccgggtaaagatgatagcgacgagcagaaa

SEQ ID NO: 3 - DNA, 3477 bp, Artificial Sequence
  Cas13a
atgaaagtgacgaaggtaggaggcatttcgcataagaagtacacgtccgaaggccgcttagtgaagtcagaatcggaagaaaatcgcacagacgaacgtctgtcggcgttgcttaatatgcgccttgacatgtatatcaagaatcccagcagcacggaaaccaaggaaaatcaaaaacgcattgggaaattaaagaaattcttctcaaacaaaatggtctatcttaaagacaataccttgagtttgaagaatgggaaaaaggagaacattgatcgtgagtattctgagactgacatccttgagagcgatgtccgtgacaagaaaaacttcgccgtgttgaaaaagatctatctgaatgaaaacgtgaactcggaggaattggaagtttttcgtaacgacattaagaagaaactgaacaaaatcaacagcctgaagtactcatttgaaaagaataaggcgaattatcaaaagattaatgagaataacatcgagaaggttgaaggtaagtcaaagcgtaacattatttacgattattatcgtgagtcagcgaaacgtgacgcttatgtaagcaatgtgaaagaagcctttgataagctttacaaggaagaggacattgcaaaacttgttcttgaaattgagaaccttacgaagttagagaaatacaagattcgcgagttctaccacgaaattattggacgtaagaatgacaaggaaaactttgcaaaaatcatctacgaagaaatccagaatgttaataacatgaaagagttgatcgagaaggtaccggacatgagtgagttgaaaaagagccaagtattttacaagtattacttagacaaagaagagttgaacgacaagaacatcaaatacgcgttttgtcatttcgtggaaatcgaaatgagtcagttgctgaagaactacgtatataagcgcttaagtaatatctcgaatgacaaaattaagcgtatctttgaataccagaacttgaaaaaattgatcgaaaataagctgttaaacaaacttgacacgtacgtccgtaattgtggaaagtataattattatttgcaagacggcgaaattgccacttcagatttcatcgcccgcaaccgtcagaatgaagcgtttcttcgcaacatcattggggtgtcatctgtggcctacttttctcttcgcaacattcttgaaacggagaacgagaatgatattactgggcgtatgcgcggcaaaacagttaagaacaataaaggtgaagagaagtacgtgtccggagaagttgataagatctataatgaaaataagaagaacgaggttaaggagaacttaaaaatgttctattcgtacgatttcaatatggacaacaagaatgaaatcgaagatttcttcgccaacatcgacgaggcgatttcttccatccgtcacggtattgtccacttcaacttggaattagaaggtaaggatatctttgcgttcaagaacattgcgccatccgaaatctcaaagaagatgtttcagaatgagattaacgagaaaaaactgaaattgaagatctttcgtcaactgaactctgccaacgtgttccgctatctcgaaaagtataaaattctgaattaccttaaacgtacacgcttcgagtttgtcaataaaaatatcccattcgtcccgtctttcaccaaattatattcgcgcattgatgacctgaagaatagtcttgggatttactggaaaactccgaaaacaaacgacgacaataagactaaggagattattgatgcccaaatctatttgcttaaaaacatctattacggggagttcctgaattatttcatgtcgaacaatggtaatttctttgagatttctaaagaaatcatcgaattgaacaagaacgataaacgcaacttaaagactgggttttacaagctgcaaaagtttgaagacatccaggagaagattccaaaggaatacttggcgaatatccagtccctgtacatgattaatgccggtaatcaggacgaagaagaaaaggacacttatattgatttcattcaaaagatcttcttaaagggatttatgacgtatcttgctaataacggtcgtttaagtctgatttacatcggctcggatgaagaaacaaatacgtcattagcagaaaagaagcaagagtttgacaagttcttgaagaagtacgagcagaacaataatatcaagatcccctatgagatcaatgaattcctgcgtgagatcaaactgggaaacatcctgaagtatactgagcgtttaaacatgttctaccttatcttaaagcttttgaatcacaaggagctgacaaatctgaagggtagtcttgaaaaatatcagtctgccaataaggaagaagcgttctctgaccaattggagttaattaacctgcttaaccttgacaacaaccgcgtgacggaagacttcgaattagaggccgacgagattggaaaatttcttgatttcaatggcaacaaagttaaggataacaaggaactgaaaaagttcgatacaaacaagatctactttgacggcgagaacattatcaaacaccgtgccttctacaatattaagaaatatggcatgttaaacttactggagaaaattgccgacaaggctggatacaagatctcgatcgaagagctgaagaaatactccaataaaaagaatgagatcgagaagaaccataagatgcaggaaaatctgcaccgcaaatacgctcgtccccgtaaagacgagaagtttacagatgaggactatgaaagttacaagcaagctattgagaatattgaggagtacacccaccttaagaacaaggtagaattcaatgagctgaatttactgcagggcctgttgctgcgcattttacatcgtttagtcggatatacctcaatttgggaacgcgatctgcgcttccgccttaaaggtgagttcccagaaaaccaatacatcgaagagatcttcaactttgaaaataagaagaacgtgaagtacaaagggggtcagattgtagagaaatacattaaattctacaaggaattacatcaaaatgatgaagttaagatcaacaagtacagttccgcgaatatcaaggtgttgaagcaagaaaagaaggacctttatattcgaaattacatcgcccacttcaattatattcctcacgccgagatctcactgctggaagtccttgaaaatttgcgtaaattgctgtcctacgatcgcaaactgaaaaatgccgtaatgaaatcagtagttgatatccttaaggagtatggttttgtagccacattcaaaatcggggcggacaagaagatcggtattcagacactggagagcgaaaaaatcgtgcatcttaagaatcttaagaagaagaagttaatgactgaccgcaattccgaggaactttgcaaattggtgaagattatgtttgaatacaaaatggaagagaaaaagtctgaaaac

SEQ ID NO: 4 - DNA, 29 bp, Artificial Sequence
  guide RNA targeting sequence
acatggtaccacatatatcacgtcaacgt

SEQ ID NO: 5 - DNA, 22 bp, Artificial Sequence
  guide RNA targeting sequence
tgaaccaagacgcagtattatt

SEQ ID NO: 6 - DNA, 31 bp, Artificial Sequence
  Cas13 recognition element
ggaccaccccaaaaatgaaggggactaaaac

(19) United States
(12) Patent Application Publication
(10) Pub. No.: US 2022/0251601 A1
(43) Pub. Date: Aug. 11, 2022

(54) mRNA NANOCAPSULE AND USE IN PREPARATION OF ANTIVIRAL DRUGS

(71) Applicant: VL Biotech Inc., New Taipei (TW)

(72) Inventor: Chiao-Long HSIAO, Taipei (TW)

(21) Appl. No.: 17/528,851

(22) Filed: Nov. 17, 2021

Related U.S. Application Data
(60) Provisional application No. 63/146,914, filed on Feb. 8, 2021.

(51) Int. Cl. C12N 15/86; C12N 9/22; C12N 15/11; A61P 31/14

(52) U.S. Cl. CPC C12N 15/86; C12N 9/22; C12N 15/11; C12N 2770/20023; C12N 2310/20; C12N 2770/20043; A61P 31/14

(57) ABSTRACT

The present invention provides an mRNA nanocapsule and use thereof, comprising a virus-like particle (VLP) formed by self-assembly of a plurality of capsid proteins (CPs), an mRNA encoding Cas13 protein, and a guide RNA. The mRNA includes a capsid protein binding tag to be encapsidated in the VLP, so that the mRNA can stably enter cells, and the Cas13 protein could be translated.

### FIELD OF THE INVENTION

The present invention relates to a drug and uses thereof, and in particular, to a technology of mRNA coated with nanocapsules for drug delivery.

### BACKGROUND OF THE INVENTION

Severe Acute Respiratory Syndrome Coronavirus Type 2 (also known as Novel Coronavirus, SARS-CoV-2) is an enveloped single-stranded RNA virus that spreads mainly in the form of droplets such as coughing or sneezing, and passes through the human respiratory tract to cause infection, inducing the symptoms such as low fever, weakness, oral and nasal symptoms, dry cough, and gastrointestinal discomfort, etc. The severe special infectious pneumonia (COVID-19) caused by SARS-CoV-2 has rapidly caused severe epidemics around the world since the end of 2019, with a total of nearly 200 million people infected and more than 4 million deaths.

As the prevention of public health at present, the common strategy against COVID-19 is vaccination around the world. However, after vaccination, it will take nearly one month to produce enough antibodies against the virus through the immune response. Furthermore, once the number of infected people accumulates in a short period of time, it will pose a pressure on the production capacity of vaccine manufacturers and the distribution of vaccines in the global community in addition to destroying the medical systems. Finally, SARS-CoV-2 has the characteristics of rapid mutation, and the numerous variants make the efficacy of existing vaccines questionable. Therefore, if drugs that can effectively treat and prevent COVID-19 can be developed, it will provide another weapon for humans fighting against the epidemics.

SARS-CoV-2, similar to common Coronaviruses, is a large and enveloped spherical single-stranded RNA virus, namely, its genetic material is ribonucleic acid (RNA). Therefore, if RNA of the SARS-CoV-2 can be destroyed, the in vivo replication and proliferation of SARS-CoV-2 can be prevented, thereby achieving the effect of treating and preventing COVID-19.

The CRISPR/Cas system is an acquired immune system found in most bacteria. It is composed of Clustered Regularly Interspaced Short Palindromic Repeats (CRISPR) and CRISPR-associated proteins (hereinafter referred to as Cas protein). Cas13 protein is an RNA nuclease that can bind with guide RNA to detect specific RNA sequences and cleave them, that is, this CRISPR/Cas system can be used to destroy RNA of the SARS-CoV-2. However, it is a problem that how to transfer this system to human cells safely and completely to achieve the above effect effectively. At present, the Cas13 system has been proved to be effective against SARS-CoV-2 and influenza virus in challenge test in animal models. However, considering the tendency of disintegration due to unstable nature of mRNA, previous experiments needed to adopt transfection methods that are toxic to cells. Moreover, Cas13 mRNA should be prepared by in vitro transcription in the past, so it is not conducive to actual clinical uses.

### SUMMARY OF THE INVENTION

In order to solve the foregoing problems, the invention provides an mRNA nanocapsule, which makes mRNA encoding a Cas13 protein (messenger RNA) to be bound to virus-like particle (VLP) and coated in the VLP to form a capsule-like structure for drug delivery.

Aiming to the above goal, the present invention provides a nucleic acid molecule, comprising a first polynucleotide sequence encoding a Cas13 protein; and a second polynucleotide sequence which identifies a VLP and includes a nucleotide sequence of SEQ ID NO: 1.

In one embodiment, the first polynucleotide sequence comprises a nucleotide sequence of SEQ ID NO: 2 or SEQ ID NO: 3.

In one embodiment, the nucleic acid molecule further comprises an internal ribosome entry site (IRES) between the first polynucleotide sequence and the second polynucleotide sequence.

The present invention further provides an mRNA nanocapsule, comprising: a virus-like particle (VLP), formed by self-assembly of capsid proteins (CPs); at least one mRNA encoding Cas13 protein, each mRNA including a capsid protein binding tag to be encapsidated in the VLP, wherein the capsid protein binding tag is encoded by SEQ ID NO: 1; and at least one guide RNA, including a targeting sequence that is reverse and complementary to a targeted site, a Cas13 protein recognition sequence, and a VLP recognition sequence including a nucleotide sequence of SEQ ID NO: 1.

In one embodiment, the plurality of CPs is selected from a group consisting of a CP of Nipah virus, Qβ, AP205 and a combination thereof.

In one embodiment, the targeting sequence of the guide RNA comprises a nucleotide sequence of SEQ ID NO: 4 or SEQ ID NO: 5.

In one embodiment, the targeted site is a nucleotide sequence derived from RNA virus, such as SARS-CoV-2, influenza viruses, etc.

In one embodiment, the targeting sequence of the guide RNA has at least 21 nucleotides.

In one embodiment, the Cas13 protein recognition sequence comprises a nucleotide sequence of SEQ ID NO: 6.

The invention further provides a use of an mRNA nanocapsule in preparation of a drug for treating novel Coronavirus disease or influenza.

In one embodiment, the novel Coronavirus disease is COVID-19.

Accordingly, in the invention, through protecting mRNA encoding a Cas13 protein by the VLP coated on the outer layer, mRNA can stably enter human cells to translate the Cas13 protein, effectively blocking the replication and proliferation of SARS-CoV-2, thus treating and preventing COVID-19 caused by SARS-CoV-2. The mRNA nanocapsule of the invention can not only overcome the shortcomings of in vitro transcription, but also can completely and safely deliver the mRNA into human cells, thereby producing the target proteins through human cells themselves and achieving the desired effect.

### BRIEF DESCRIPTION OF THE DRAWINGS

FIG. 1 is a schematic diagram of a nucleic acid molecule according to an embodiment of the present invention.

FIG. 2 is a schematic diagram of an mRNA nanocapsule according to an embodiment of the present invention.

FIG. 3 is a schematic diagram of a guide RNA according to an embodiment of the present invention.

FIG. 4 is a schematic diagram of a composition comprising an mRNA nanocapsule according to an embodiment of the present invention.

FIG. 5 is a schematic diagram of a composition comprising an mRNA nanocapsule according to another embodiment of the present invention.

FIG. 6A and FIG. 6B show experimental results of Test Example 1 of the present invention.

FIG. 7A and FIG. 7B show experimental results of Test Example 2 of the present invention.

FIG. 8A and FIG. 8B show experimental results of Test Example 3 of the present invention.

FIG. 9A and FIG. 9B show experimental results of Test Example 4 of the present invention.

FIG. 10A and FIG. 10B show experimental results of Test Example 5 of the present invention.

### DETAILED DESCRIPTION OF THE PREFERRED EMBODIMENTS

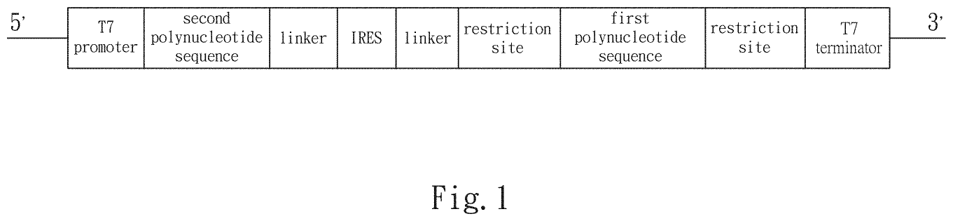

Referring to FIG. 1, the invention provides a nucleic acid molecule comprising Cas13 protein encoding segment, wherein Cas13 protein is a nuclease used in the CRISPR/Cas system to cleave single-stranded RNA. In one embodiment, the nucleic acid molecule is deoxyribonucleic acid (DNA). The nucleic acid molecule comprises a first polynucleotide sequence and a second polynucleotide sequence. The first polynucleotide sequence is a nucleotide sequence encoding Cas13 protein, and the second polynucleotide sequence is used to identify a virus-like particle (VLP), including a nucleotide sequence of SEQ ID NO: 1. The VLP referred to in the invention is a virus-like structure formed by self-assembly of a plurality of capsid proteins (CPs), and the VLP is a hollow nanostructure without viral nucleic acid. In one embodiment, the VLP is a spheroid composed of 180 CPs. In one embodiment, the plurality of CPs used in the invention is derived from bacteriophage, for example, CPs of Nipah virus, Qβ, AP205 or a combination thereof. In one embodiment, the diameter of the Qβ-VLP is about 24 nanometers, and the diameter of the AP205-VLP is about 30 nanometers.

In one embodiment, the first polynucleotide sequence includes a nucleotide sequence of SEQ ID NO: 2 encoding Cas13d protein; in another embodiment, the first polynucleotide sequence includes a nucleotide sequence of SEQ ID NO: 3 encoding Cas13a protein.

In one embodiment, the nucleic acid molecule further comprises an internal ribosome entry site (IRES) between the first polynucleotide sequence and the second polynucleotide sequence.

In one embodiment, the nucleic acid molecule further comprises two restriction sites located upstream and downstream of the first polynucleotide sequence. The two restriction sites are sequences that can be recognized by any restriction enzyme, such as EcoRI, BamHI, HindIII, XbaI, etc., but are not limited thereto.

In one embodiment, the nucleic acid molecule further comprises a promoter located at 5′ end and a terminator at 3′ end of the first polynucleotide sequence for the transcription of the RNA polymerase. In the invention, T7 promoter and T7 terminator that are recognized by T7 RNA polymerase are used, but are not limited thereto.

In one embodiment, the nucleic acid molecule further comprises two linkers located upstream and downstream of the IRES, and the linker is any polynucleotide sequence of 15 to 30 nucleotides in length.

When the nucleic acid molecule is transfected into cells, RNA polymerase recognizes the promoter on the nucleic acid molecule and starts transcription to form the corresponding mRNA which encodes the Cas13 protein.

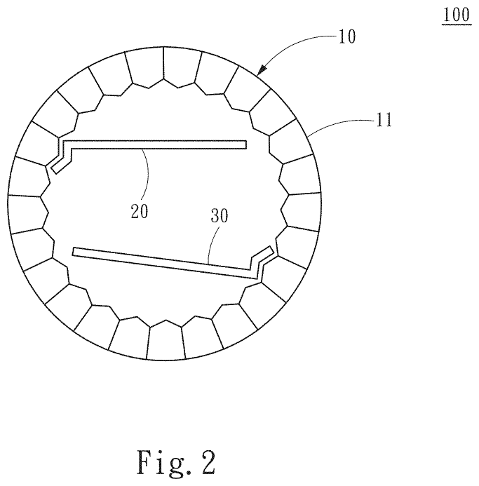

Referring to FIG. 2, it is a schematic diagram of an mRNA nanocapsule 100 provided by an embodiment of the present invention. The mRNA nanocapsule 100 is formed by encapsulating the in vivo transcribed mRNA in a nanoscale RNA-protein complex structure. The mRNA nanocapsule 100 includes a VLP 10, at least one mRNA 20 and at least one guide RNA 30. The VLP 10 is formed by self-assembly of at least one CP 11. Each of the at least one mRNA 20 is a polynucleotide encoding the Cas13 protein, and includes a capsid protein binding tag. The capsid protein binding tag is encoded by SEQ ID NO: 1, and is bound to a specific region on the CP of the VLP 10, so that the at least one mRNA 20 is encapsidated in the VLP 10. Each of the at least one guide RNA 30 includes a targeting sequence that is reverse and complementary to a targeted site of the virus, a Cas13 protein recognition sequence, and a VLP recognition sequence including a nucleotide sequence of SEQ ID NO: 1. The ratio of the number of moles of at least one mRNA 20 to the at least one guide RNA 30 is determined according to the amount and type of viruses infected or different VLPs, thereby achieving an optimized antiviral effect. In one embodiment, the number of moles of the at least one mRNA 20 is less than or equal to that of the at least one guide RNA 30. For example, the ratio of the number of moles of the at least one mRNA 20 to the at least one guide RNA 30 is 1:5, but is not limited thereto.

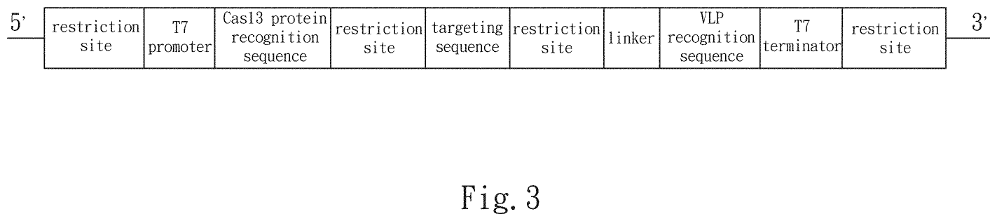

Referring to FIG. 3, it is a schematic diagram of the at least one guide RNA 30 of the invention. The Cas13 protein recognition sequence, the targeting sequence, and the VLP recognition sequence are shown sequentially from the 5′ end to the 3′ end of the guide RNA 30.

In one embodiment, the targeted site is the viral genome, and the targeting sequence is reverse and complementary to a specific segment in the RNA sequence of the virus. In one embodiment, the targeted site is a nucleotide sequence derived from SARS-CoV-2; the targeting sequence includes a nucleotide sequence of SEQ ID NO: 4 or SEQ ID NO: 5. In one embodiment, the targeting sequence comprises at least 21 nucleotides.

The Cas13 protein recognition sequence is used to bind to a specific region of the Cas13 protein and guide the Cas13 protein to the targeted site to cleave the virus RNA. In one embodiment, the Cas13 protein recognition sequence includes a nucleotide sequence of SEQ ID NO: 6.

In one embodiment, the guide RNA 30 further includes a promoter at the upstream of the Cas13 protein recognition sequence, and a terminator at the downstream of the VLP recognition sequence. In the invention, T7 promoter and T7 terminator that are recognized by T7 RNA polymerase are used, but are not limited thereto.

In one embodiment, the guide RNA 30 further includes a first pair of restriction sites located at two ends of the guide RNA 30, and a second pair of restriction sites located at the upstream and downstream of the targeting sequence. The two pairs of restriction sites may be the same or different, and can be recognized by any restriction enzymes, such as EcoRI, BamHI, HindIII, XbaI, etc., but are not limited thereto.

In one embodiment, the guide RNA 30 further includes a linker located upstream of the VLP recognition sequence, and the linker is any polynucleotide sequence of 15 to 30 nucleotides in length.

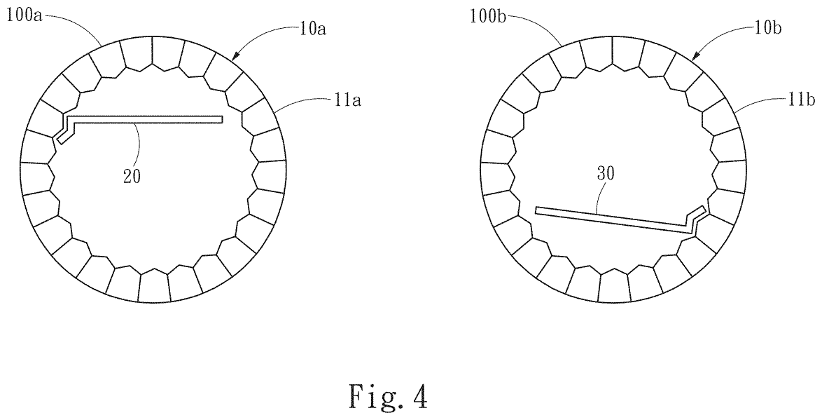

Referring to FIG. 4, it is a composition comprising an mRNA nanocapsule according to an embodiment of the present invention. The composition includes a plurality of mRNA nanocapsules 100a and a plurality of guide RNA nanocapsules 100b. Each of the plurality of mRNA nanocapsules 100a includes a first VLP 10a and at least one mRNA 20 encoding Cas13 protein. The first VLP 10a is formed by self-assembly of a plurality of first CPs 11a. Each of the plurality of guide RNA nanocapsules 100b includes a second VLP 10b and at least one guide RNA 30, and the second VLP 10b is formed by self-assembly of a plurality of second CPs 11b. The structures of the mRNA 20 and the guide RNA 30 are the same as those in the previous embodiment, and will not be described again herein. The first CPs 11a and the second CPs 11b may be the same or different. The ratio of the number of moles of the mRNA nanocapsules 100a to the guide RNA nanocapsules 100b is determined according to the amount and type of viruses infected or different VLPs, thereby achieving an optimized antiviral effect. In one embodiment, the ratio of the number of moles of the mRNA nanocapsules 100a to the guide RNA nanocapsules 100b is between 1:10 and 1:30, for example, the ratio of the number of moles is 1:20, but is not limited thereto.

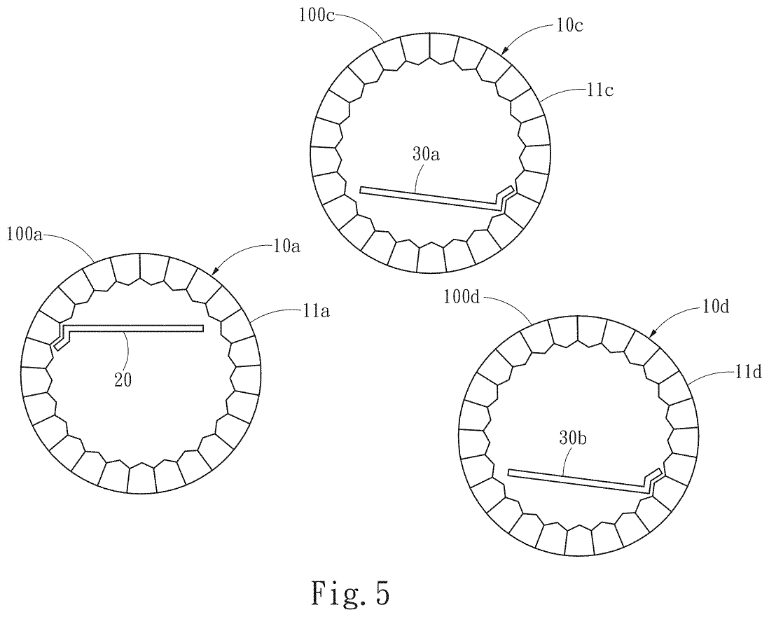

Referring to FIG. 5, it is a composition comprising an mRNA nanocapsule according to another embodiment of the present invention. The composition includes a plurality of mRNA nanocapsules 100a, a plurality of first guide RNA nanocapsules 100c, and a plurality of second guide RNA nanocapsules 100d. Each of the plurality of mRNA nanocapsules 100a includes a first VLP 10a and at least one mRNA 20 encoding Cas13 protein. The first VLP 10a is formed by self-assembly of a plurality of first CPs 11a. Each of the plurality of first guide RNA nanocapsules 100c includes a third VLP 10c and at least one first guide RNA 30a, and the third VLP 10c is formed by self-assembly of a plurality of third CPs 11c. Each of the second guide RNA nanocapsules 100d includes a fourth VLP 10d and at least one second guide RNA 30b, and the fourth VLP 10d is formed by self-assembly of a plurality of fourth CPs 11d. The first guide RNA 30a and the second guide RNA 30b respectively comprise targeting sequences with different nucleotide sequences. In one embodiment, the targeting sequence in the first guide RNA 30a includes a nucleotide sequence of SEQ ID NO: 4, and the targeting sequence in the second guide RNA 30b includes a nucleotide sequence of SEQ ID NO: 5. The plurality of first CPs 11a, the plurality of third CPs 11c, and the plurality of fourth CPs 11d may be the same or different. The structures of the mRNA 20, the first guide RNA 30a, and the second guide RNA 30b are the same as those in the foregoing embodiments, and will not be described again herein.

The invention further provides a use of the mRNA nanocapsule in preparation of a drug for treating or preventing SARS-CoV-2. When the mRNA nanocapsule 100 enters the SARS-CoV-2 infected cell, the Cas13 protein is translated, and the targeted site derived from the nucleotide sequence of SARS-CoV-2 is bound with the guide RNA since the targeted site is complementary to the targeting sequence of the guide RNA, thereby guiding the Cas13 protein to cleave the targeted site.

The following examples are only used to illustrate the purpose of the invention, but not limit the scope of the invention. Those skilled in the art can produce other specific embodiments, substitutions and changes according to the disclosure and teachings of the present invention.

### [Example 1] Preparation of Target Vector

An RNA segment of SARS-CoV-2 gene was inserted into a green fluorescent protein (GFP) expression plasmid as the target vector to be cleaved in the present invention.

### [Example 2] Preparation of Capsule Vector

A nucleotide encoding CP was inserted into a plasmid as a capsule vector for the production of VLP.

### [Example 3] Preparation of Cas Vector

A nucleotide identifying the VLP and a nucleotide encoding the Cas13 protein were inserted into a plasmid as a Cas vector for cleaving the target vector of Example 1.

### [Example 4] Preparation of Guide RNA Vector

A nucleotide identifying the VLP and a nucleotide encoding the guide RNA were inserted into a plasmid as a guide RNA vector for identifying the target vector of Example 1.

### [Example 5] Preparation of Nanocapsules

The capsule vector of Example 2, the Cas vector of Example 3, and the guide RNA vector of Example 4 were transformed into Escherichia coli, so that the translation of CP and the transcription of Cas13 mRNA or guide RNA were carried out simultaneously in Escherichia coli. Cas13 mRNA and guide RNA bound the VLP formed by self-assembly of CPs through the nucleotide identifying the VLP, so as to spontaneously assemble into nanocapsules.

### [Test Example 1] Therapeutic Effect of mRNA Nanocapsules on COVID

The target vector of Example 1 was transfected into human embryonic kidney cells (HEK293), and the untransfected vector was washed away with PBS buffer. Then, the nanocapsules of Example 5 were added to HEK293 cells, so that the mRNA and the guide RNA in the nanocapsules were transfected into HEK293 cells. The transfected cells were cultured for 4 hours, 10 hours, and 21 hours. Fluorescence images were captured by a fluorescence microscope, and the fluorescence values were analyzed by image analysis software.

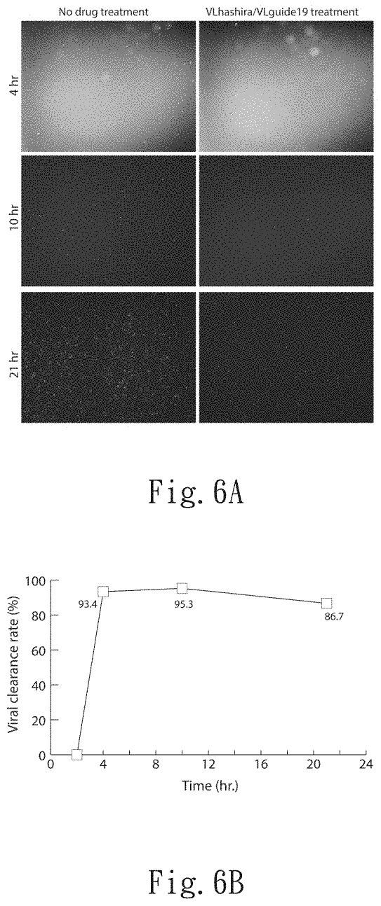

FIG. 6A and FIG. 6B showed the experimental results of this test example. In FIG. 6A, the left column showed the fluorescence image of the cells of the control group that was not treated with the mRNA nanocapsules. The right column showed the fluorescence image of the cells of the experimental group treated with the mRNA nanocapsules. After 21 hours of culture, the amount of fluorescence of the cells in the experimental group was significantly lower than that of the control group, i.e. the mRNA nanocapsules significantly reduced the amount of the target vector which comprises the RNA segment of SARS-CoV-2 gene in the cells. The fluorescence values were analyzed by the image analysis software and the viral clearance rates were calculated, as shown in FIG. 6B, after 10 hours of culture, the viral clearance rate of the experimental group treated with the mRNA nanocapsules was greater than 90%; and after 21 hours of culture, the viral clearance rate could still maintain at 86.7%. The experimental results showed that the mRNA nanocapsules of the invention were therapeutically effective to COVID-19 caused by SARS-CoV-2.

### [Test Example 2] Preventive Effect of Multi-Dose mRNA Nanocapsules on SARS-CoV-2

The nanocapsules of Example 5 were first added to HEK293 cells in multiple doses, so that the mRNA and the guide RNA in the nanocapsules were transfected into HEK293 cells, and the untransfected nanocapsules was removed; then, the target vector of Example 1 was transfected into HEK293 cells, and the untransfected vector was washed away with PBS buffer.

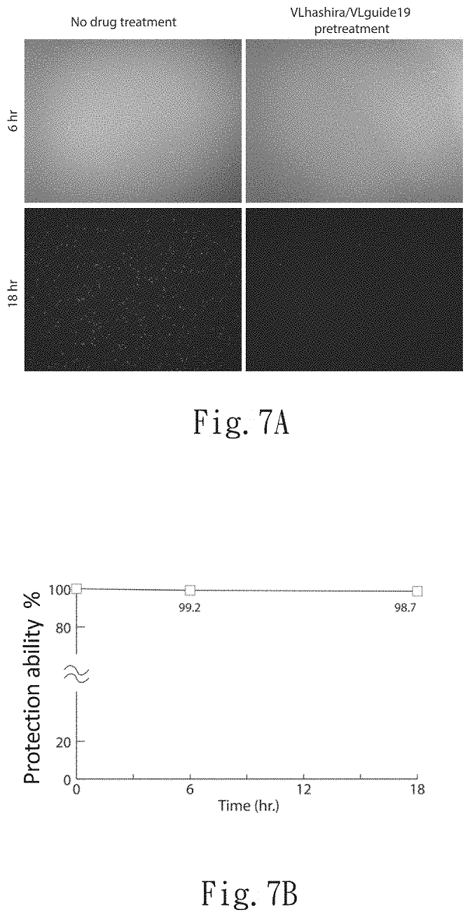

FIG. 7A and FIG. 7B showed the experimental results of this test example. In FIG. 7A, the left column showed the fluorescence image of the cells of the control group that was not treated with the mRNA nanocapsules. The right column showed the fluorescence image of the cells of the experimental group treated with the mRNA nanocapsules. After 18 hours of culture, the amount of fluorescence of the cells in the experimental group was significantly lower than that of the control group, as shown in FIG. 7B, the protection ability of the experimental group with pre-treated mRNA nanocapsules was still close to 100% after 18 hours. Thus, according to the experimental results, the mRNA nanocapsules pre-treated to the cells were effective to prevent COVID-19 caused by SARS-CoV-2.

### [Test Example 3] Preventive Effect of Single-Dose mRNA Nanocapsule on SARS-CoV-2

The nanocapsules of Example 5 were first added to HEK293 cells a single dose, so that the mRNA and guide RNA in the nanocapsules were transfected into HEK293 cells, and the untransfected nanocapsules was removed; then, the target vector of Example 1 was transfected into HEK293 cells, and the untransfected vector was washed away with PBS buffer.

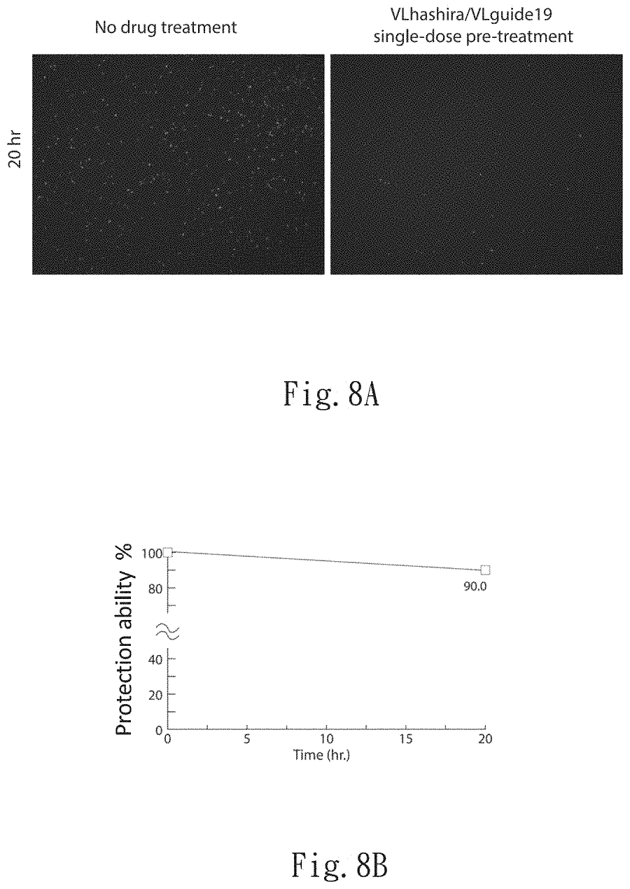

FIG. 8A and FIG. 8B showed the experimental results of this test example. In FIG. 8A, the left column showed the fluorescence image of the cells of the control group that was not treated with the nanocapsules. The right column showed the fluorescence image of the cells of the experimental group treated with the nanocapsules. After 20 hours of culture, the amount of fluorescence of the cells in the experimental group was significantly lower than that of the control group, as shown in FIG. 8B, the protection ability in the experimental group with pre-treated mRNA nanocapsules was still close to 90% after 20 hours. Thus, according to the experimental results, the mRNA nanocapsules pre-treated to the cells were effective to prevent to COVID-19 caused by SARS-CoV-2.

### [Test Example 4] mRNA Nanocapsule could Quickly Adapt to Viral RNA Mutations

In this test example, natural GFP expression plasmids without RNA segment of SARS-CoV-2 gene were transfected into HEK293 cells as mutations of SARS-CoV-2 RNA. Then, the nanocapsules of the invention were added to HEK293 cells. The difference from the above test examples lies in the arrangement that the targeting sequence of the guide RNA used in this test example was reverse and complementary to nucleotide sequence of the natural GFP expression plasmid.

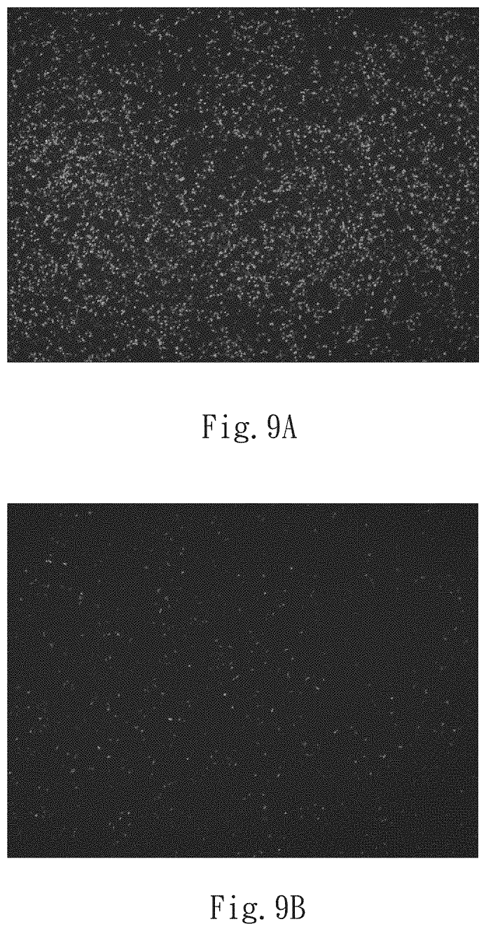

FIG. 9A and FIG. 9B showed the experimental results of this test example. FIG. 9A showed the fluorescence image of the cells of the control group that was not treated with the mRNA nanocapsules. FIG. 9B showed the fluorescence image of the cells of the experimental group treated with the mRNA nanocapsules. Comparing FIG. 9A with FIG. 9B, the amount of fluorescence of the cells in the experimental group was significantly lower than that of the control group, indicating that the mRNA nanocapsules of the invention could quickly adapt to the infection of viral RNA mutations.

### [Test Example 5] Specificity of mRNA Nanocapsules

In this test example, the natural GFP expression plasmids were transfected into HEK293 cells, and then the nanocapsules of the invention were added into HEK293 cells, wherein the targeting sequence of the guide RNA was the reverse and complementary to SARS-CoV-2 (not the natural GFP expression plasmid).

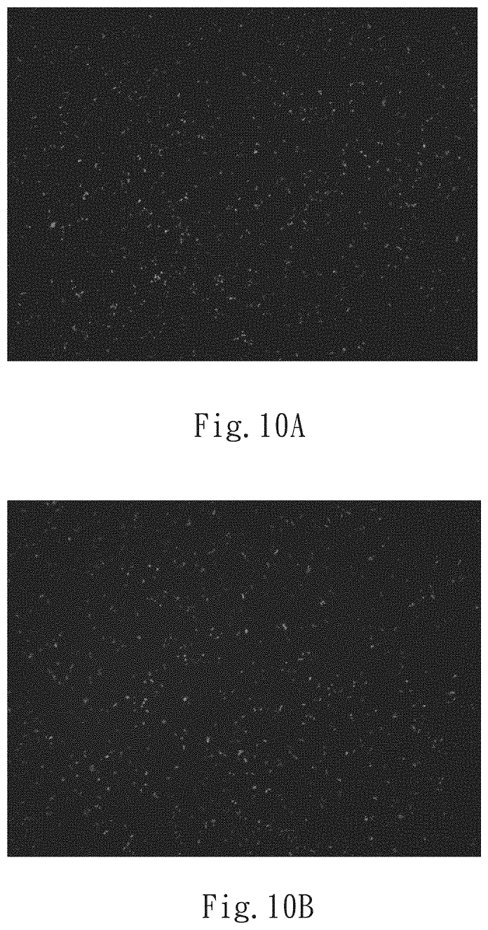

FIG. 10A and FIG. 10B showed the experimental results of this test example. FIG. 10A showed the fluorescence image of the cells of the control group that was not treated with the mRNA nanocapsules. FIG. 10B showed the fluorescence image of the cells of the experimental group treated with the mRNA nanocapsules. Comparing FIG. 10A with FIG. 10B, the amount of fluorescence of the cells in the experimental group was close to that of the control group, indicating that the guide RNA comprising the targeting sequence that was reverse and complementary to SARS-CoV-2 could not recognize the natural GFP expression plasmids, thus could not guide the Cas13 protein to cleave the natural GFP expression plasmids. The experimental results showed that the mRNA nanocapsules of the invention have specificity through the designed targeting sequence.

What is claimed is:

1. A nucleic acid molecule, comprising:
a first polynucleotide sequence encoding Cas13 protein; and
a second polynucleotide sequence identifying a virus-like particle (VLP), including a nucleotide sequence of SEQ ID NO: 1.

2. The nucleic acid molecule according to claim 1, wherein the first polynucleotide sequence comprises a nucleotide sequence of SEQ ID NO: 2 or SEQ ID NO: 3.

3. The nucleic acid molecule according to claim 1, wherein the nucleic acid molecule further comprises an internal ribosome entry site (IRES) between the first polynucleotide sequence and the second polynucleotide sequence.

4. An mRNA nanocapsule, comprising:
a virus-like particle (VLP), formed by self-assembly of a plurality of capsid proteins (CPs);
at least one mRNA encoding Cas13 protein, each mRNA including a capsid protein binding tag to be encapsidated in the VLP, wherein the capsid protein binding tag is encoded by SEQ ID NO: 1; and
at least one guide RNA, including a targeting sequence reverse and complementary to a targeted site, a Cas13 protein recognition sequence, and a VLP recognition sequence including a nucleotide sequence of SEQ ID NO: 1.

5. The mRNA nanocapsule according to claim 4, wherein the plurality of CPs is selected from a group consisting of a CP of Nipah virus, Qβ, AP205 and a combination thereof.

6. The mRNA nanocapsule according to claim 4, wherein the targeting sequence of the guide RNA comprises a nucleotide sequence of SEQ ID NO: 4 or SEQ ID NO: 5.

7. The mRNA nanocapsule according to claim 4, wherein the targeted site is a viral genome.

8. The mRNA nanocapsule according to claim 5, wherein the Cas13 protein recognition sequence comprises a nucleotide sequence of SEQ ID NO: 6.

9. The mRNA nanocapsule according to claim 4, wherein a number of moles of the at least one mRNA is less than or equal to that of the at least one guide RNA.

10. A composition comprising an mRNA nanocapsule, comprising:
a plurality of mRNA nanocapsules, each of the plurality of mRNA nanocapsules including a first virus-like particle (VLP) formed by self-assembly of a plurality of first capsid proteins (CPs) and at least one mRNA encoding Cas13 protein, wherein each mRNA includes a capsid protein binding tag to be encapsidated in the first VLP, and the capsid protein binding tag is encoded by SEQ ID NO: 1; and
a plurality of guide RNA nanocapsules, each of the plurality of guide RNA nanocapsules including a second VLP formed by self-assembly of a plurality of second CPs and at least one guide RNA, wherein each guide RNA includes a targeting sequence that is reverse and complementary to a targeted site, a Cas13 protein recognition sequence, and a VLP recognition sequence including a nucleotide sequence of SEQ ID NO: 1.

11. The composition according to claim 10, wherein a ratio of a number of moles of the plurality of mRNA nanocapsules to the plurality of guide RNA nanocapsules is between 1:10 and 1:30.

12. The composition according to claim 10, wherein the first CP and the second CP are selected from a group consisting of a CP of Nipah virus, Qβ, AP205 and a combination thereof.

13. The composition according to claim 10, wherein the targeting sequence of the guide RNA comprises a nucleotide sequence of SEQ ID NO: 4 or SEQ ID NO: 5.

14. The composition according to claim 10, wherein the targeted site is a viral genome.

15. The composition according to claim 10, wherein the Cas13 protein recognition sequence comprises a nucleotide sequence of SEQ ID NO: 6.

16. A use of the mRNA nanocapsule according to claim 4 in preparation of antiviral drugs.

17. A use of the mRNA nanocapsule according to claim 4 in preparation of a drug for treating novel Coronavirus disease or influenza.
